SEQUENCE LISTING (1 sequence)

SEQ ID NO: 1 - protein, 1668 aa, Bacillus subtilis
AGCTCGGATCCACTAGTAACGGCCGCCAGTGTGCTGGAATTCGCCCTTAGAAAGGAGGTGATCCAGCCGCACCTTCCGATACGGCTACCTTGTTACGACTTCACCCCAATCATCTGTCCCACCTTCGGCGGCTGGCTCCATAAAGGTTACCTCACCGACTTCGGGTGTTACAAACTCTCGTGGTGTGACGGGCGGTGTGTACAAGGCCCGGGAACGTATTCACCGCGGCATGCTGATCCGCGATTACTAGCGATTCCAGCTTCACGCAGTCGAGTTGCAGACTGCGATCCGAACTGAGAACAGATTTGTGRGATTGGCTTAACCTCGCGGTTTCGCTGCCCTTTGTTCTGTCCATTGTAGCACGTGTGTAGCCCAGGTCATAAGGGGCATGATGATTTGACGTCATCCCCACCTTCCTCCGGTTTGTCACCGGCAGTCACCTTAGAGTGCCCAACTGAATGCTGGCAACTAAGATCAAGGGTTGCGCTCGTTGCGGGACTTAACCCAACATCTCACGACACGAGCTGACGACAACCATGCACCACCTGTCACTCTGCCCCCGAAGGGGACGTCCTATCTCTAGGATTGTCAGAGGATGTCAAGACCTGGTAAGGTTCTTCGCGTTGCTTCGAATTAAACCACATGCTCCACCGCTTGTGCGGGCCCCCGTCAATTCCTTTGAGTTTCAGTCTTGCGACCGTACTCCCCAGGCGGAGTGCTTAATGCGTTAGCTGCAGCACTAAAGGGGCGGAAACCCCCTAACACTTAGCACTCATCGTTTACGGCGTGGACTACCAGGGTATCTAATCCTGTTCGCTCCCCACGCTTTCGCTCCTCAGCGTCAGTTACAGACCAGAGAGTCGCCTTCGCCACTGGTGTTCCTCCACATCTCTACGCATTTCACCGCTACACGTGGAATTCCACTCTCCTCTTCTGCACTCAAGTTCCCCAGTTTCCAATGACCCTCCCCGGTTGAGCCGGGGGCTTTCACATCAGACTTAAGAAACCGCCTGCGAGCCCTTTACGCCCAATAATTCCGGACAACGCTTGCCACCTACGTATTACCGCGGCTGCTGGCACGTAGTTAGCCGTGGCTTTCTGGTTAGGTACCGTCAAGGTGCCGCCCTATTTGAACGGCACTTGTTCTTCCCTAACAACAGAGCTTTACGATCCGAAAACCTTCATCACTCACGCGGCGTTGCTCCGTCAGACTTTCGTCCATTGCGGAAGATTCCCTACTGCTGCCTCCCGTAGGAGTCTGGGCCGTGTCTCAGTCCCAGTGTGGCCGATCACCCTCTCAGGTCGGCTACGCATCGTCGCCTTGGTGAGCCGTTACCTCACCAACTAGCTAATGCGCCGCGGGTCCATCTGTAAGTGGTAGCCGAAGCCACCTTTTATGTCTGAACCATGCGGTTCAGACAACCATCCGGTATTAGCCCCGGTTTCCCGGAGTTATCCCAGTCTTACAGGCAGGTTACCCACGTGTTACTCACCCGTCCGCCGCTAACATCAGGGAGCAAGCTCCCATCTGTCCGCTCGACTTGCATGTATTAGGCACGCCGCCAGCGTTCGTCCTGAGCCATGAACAAACTCTAAGGGCGAATTCTGCAGATATCCATCACACTGGCGGCCGCTCGAGCATGCATCTAGAGGGCCCAATCGCCCTAT

(19) United States
(12) Patent Application Publication
(10) Pub. No.: US 2016/0312252 A1
(43) Pub. Date: Oct. 27, 2016

(54) COMPOSITIONS AND METHODS FOR BIODIESEL PRODUCTION FROM WASTE TRIGLYCERIDES

(71) Applicant: BiOWiSH Technologies, Inc., Cincinnati, OH (US)

(72) Inventors: Richard S. Carpenter, West Chester, OH (US); Irawan Hartantio, Jakarta Barat (ID)

(21) Appl. No.: 15/137,768

(22) Filed: Apr. 25, 2016

Related U.S. Application Data
(60) Provisional application No. 62/152,471, filed on Apr. 24, 2015.

(51) Int. Cl. C12P 7/64; C10L 1/02

(52) U.S. Cl. CPC C12P 7/649; C10L 1/026; C10L 2200/0476; C10L 2270/026; C10L 2290/26

(57) ABSTRACT

The present invention relates to a process for creating biodiesel from triglyceride waste.

### RELATED APPLICATIONS

This application claims priority to and benefit of U.S. Provisional Application No. 62/152,471, filed on Apr. 24, 2015, the contents of which are hereby incorporated by reference in their entireties.

### INCORPORATION-BY-REFERENCE OF SEQUENCE LISTING

The contents of the text file named “BIOW-012-001WO-Sequence Listing.txt”, which was created on Apr. 25, 2016 and is 14.4 KB in size, are hereby incorporated by reference in their entireties.

### FIELD OF THE INVENTION

The present invention relates to biodiesel production compositions containing micro-organisms and methods of using the compositions.

### BACKGROUND OF THE INVENTION

Biodiesel has physio-chemical properties that are similar to those of petroleum-based diesel. Because of its renewability, biodiesel has attracted interest from oil and chemical companies as well as newly emerged alternative fuel companies. The conventional process used for biodiesel production is cost intensive, which is partly attributed to the transesterification reaction. In addition, despite numerous environmental benefits compared with petroleum-based diesel, the conventional biodiesel production also has some environmental challenges. For example, methanol, which is routinely used in the transesterification reaction, can be hazardous. When removing residual triglycerides and glycerol from the biodiesel product, multiple steps of water wash produce massive industrial wastewater that can have tremendous negative impact on the environment. Therefore, novel strategies for biodiesel production are highly sought by the industry.

Biodiesel is a mixture of fatty acid methyl esters (FAMEs) that are derived from a variety of crop oils, animal fats or waste oils. In the conventional process of biodiesel production, transesterification is a critical step. It utilizes basic or acid catalysts to convert triglycerides into FAMEs in the presence of methanol with glycerol as a by-product. The existence of glycerol has been proved to affect the quality of biodiesel, such as viscosity, flash point and oxidation stability. Therefore, glycerol has to be removed.

The transesterification reaction itself also has a number of technical challenges, such as the low reaction rate when using the acid catalyst and the formation of soap in basic-based process. There have been numerous attempts focusing on improving the conventional transesterification process for biodiesel production. For example, several studies reported utilizing lipase as a catalyst to catalyze the removal of glycerol and the formation of FAMEs. In a recent report, methyl acetate, instead of methanol, was used in the transesterification reaction in order to reduce the influence of glycerol. While some progress has been made in improvement of biodiesel production, break-through concepts and technologies still need to be developed in order to significantly lower the cost of biodiesel production to make it economically feasible.

### SUMMARY OF THE INVENTION

In various aspects the invention provides methods of converting triglyceride containing waste into biodiesel by combining in a reactor triglyceride containing waste, alcohol (e.g., methanol or ethanol) and a microbial biocatalyst comprising a mixture of Bacillus and Lactobacillus organisms and subjecting the resulting mixture to sonication. Optionally, the resulting biodiesel is washed with water to remove traces of the microbial catalyst and any unreacted alcohol. The volume of triglyceride containing waste material comprises from 50-90% of the useable volume of the reactor. The alcohol concentration ranges from 10-15% by weight of the triglyceride containing waste material. Preferably, the microbial catalyst is added at 0.01 to 1.5% by weight of the triglyceride containing waste material triglyceride containing waste material. The sonication is conducted for 5-20 minutes. The triglyceride waste is derived from used cooking oil, sludge palm oil, palm, rapeseed, soybean, mustard, flax, sunflower, canola, hemp, jatropha or mixtures thereof.

The Bacillus organisms are a mixture of Bacillus subtilis, Bacillus amyloliquefaciens, Bacillus licheniforms, and Bacillus pumilus. Optionally, the Bacillus organisms further comprise a mixture of Bacillus megaterium, Bacillus coagulans, and Paenibacillus polymyxa. In some embodiments each of the Bacillus in the mixture is individually aerobically fermented, harvested, dried, and ground to produce a powder having a mean particle size of about 200 microns, with greater than about 60% of the mixture in the size range between 100-800 microns.

The Lactobacillus organisms are a mixture of Pediococcus acidilactici, Pediococcus pentosaceus, and Lactobacillus plantarum. In some embodiments each of the Lactobacillus in the mixture is individually anaerobically fermented, harvested, dried, and ground to produce a powder having a mean particle size of about 200 microns, with greater than about 60% of the mixture in the size range between 100-800 microns.

The ratio of the Bacillus to Lactobacillus is between 1:10 to 10:1. The microbial biocatalyst has a moisture content of less than about 5%; and a final bacterial concentration of about between 10⁵-10¹¹ colony forming units (CFU) per gram. In some aspects, the microbial biocatalyst further comprising an inert carrier such as rice bran, soybean meal, wheat bran, dextrose monohydrate, anhydrous dextrose, maltodextrin, or a mix thereof. The inert carrier is at a concentration of about 75-95% (w/w).

In another aspect, the microbial biocatalyst further comprises an organic emulsifier such as soy lecithin. The organic emulsifier is at a concentration of about between 1 to 5% (w/w).

In one aspect, the microbial biocatalyst comprises about 87.9% by weight of dextrose, about 1% by weight of Bacillus Mix #1, about 1% by weight of Bacillus Mix #2, about 0.1% Bacillus Mix #3 and 10% by weight of Lactobacillus Mix #1.

In another aspect, the microbial biocatalyst comprises about 2.1% a Bacillus mixture by weight, about 10% a Lactobacillus mixture by weight and about 87.9% dextrose by weight. The Bacillus mixture comprises 30% Bacillus subtilis by weight, about 20% Bacillus amyloliquefaciens by weight, about 30% Bacillus licheniformis by weight, and about 20% Bacillus pumilus by weight. The Lactobacillus mixture includes equal amounts of Pediococcus acidilactici, Pediococcus pentosaceus and Lactobacillus plantarum by weight.

In various aspects, the invention provides a composition comprising about 2.1% a Bacillus mixture by weight, about 10% a Lactobacillus mixture by weight and about 87.9% dextrose by weight, wherein the Bacillus mixture comprises about 30% Bacillus subtilis by weight, about 20% Bacillus amyloliquefaciens by weight, about 30% Bacillus licheniformis by weight, and about 20% Bacillus pumilus by weight, and wherein the Lactobacillus mixture comprises equal amounts of Pediococcus acidilactici, Pediococcus pentosaceus and Lactobacillus plantarum by weight. The composition disclosed herein can be used as a microbial biocatalyst for converting triglyceride containing waste into biodiesel.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a schematic of the ultrasonic continuous biodiesel production process of the invention.

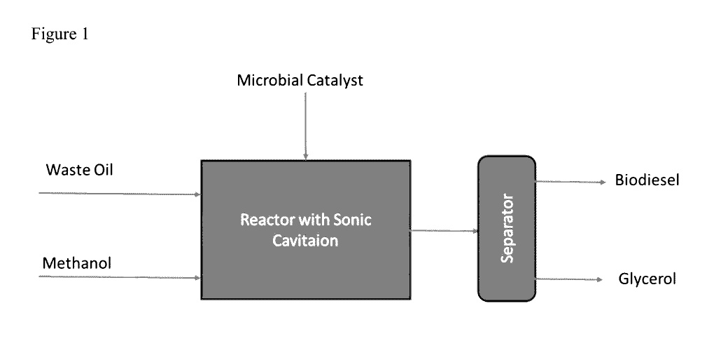

### DETAILED DESCRIPTION OF THE INVENTION

Biodiesel is a time intensive production process. Finding ways to shorten the production time can greatly impact the feasibility of large scale industrial production. The present methods utilize a microbial catalyst addition and sonication to achieve significant reductions in the time taken to produce biodiesel. The combination of the microbial catalyst and sonication achieved greater than 95% conversion of triglyceride waste to biodiesel in ten minutes or less at room temperature. The invention further provides microbial compositions for augmenting the conversion of triglyceride containing waste material into biodiesel. Additionally, the invention provides methods for using the microbial composition as a microbial catalyst to produce biodiesel.

The term “microbial” “bacteria” or “microbes” as used herein, refers to microorganisms that confer a benefit. The microbes according to the invention may be viable or non-viable. The non-viable microbes are metabolically-active. By “metabolically-active” is meant that they exhibit at least some residual enzyme, or secondary metabolite activity characteristic to that type of microbe.

By the term “non-viable” as used herein is meant a population of bacteria that is not capable of replicating under any known conditions. However, it is to be understood that due to normal biological variations in a population, a small percentage of the population (i.e., 5% or less) may still be viable and thus capable of replication under suitable growing conditions in a population which is otherwise defined as non-viable.

By the term “viable bacteria” as used herein is meant a population of bacteria that is capable of replicating under suitable conditions under which replication is possible. A population of bacteria that does not fulfill the definition of “non-viable” (as given above) is considered to be “viable”.

As used herein, the term “about” in conjunction with a numeral refers to the numeral and a deviation thereof in the range of ±10% of the numeral. For example, the phrase “about 100” refers to a range of 90 to 110.

Unless stated otherwise, all percentages mentioned in this document are by weight based on the total weight of the composition.

The microbes used in the product according to the present invention may be any conventional mesophilic bacteria. It is preferred that the bacteria are selected from the Lactobacillacae and Bacillaceae families. More preferably the bacteria selected form the genus Bacillus and Lactobacillis are included in the compositions of the invention. The bacterial compositions of the invention are used as a biocatalyst to facilitate the conversation of triglyceride containing waste into biodiesel.

A preferred microbial biocatalyst according to the invention includes about 85% to 95% by weight of dextrose and the remainder by weight of a microbial mixture. Preferably, the microbial mixture includes a Bacillus mixture and a Lactobacillus mixture. The dextrose can be dextrose monohydrate, anhydrous dextrose or a combination thereof

The Bacillus mixture includes Bacillus subtilis, Bacillus amyloliquefaciens, Bacillus licheniformis, and Bacillus pumilus. Optionally the Bacillus mixture further includes Bacillus coagulans, Bacillus megaterium, and Paenibacillus polymyxa. The Bacillus subtilis can include Mojavensis. The Bacillus subtilis can include Bacillus subtilis 34KLB. The Lactobacillus mixture includes Pediococcus acidilactici, Pediococcus pentosaceus and Lactobacillus plantarum.

The amino acid sequence of Bacillus subtilis 34KLB is shown below:

Bacillus subtilis strain 34KLB
(SEQ ID NO: 1)
AGCTCGGATCCACTAGTAACGGCCGCCAGTGTGCTGGAATTCGCCCTTAG
AAAGGAGGTGATCCAGCCGCACCTTCCGATACGGCTACCTTGTTACGACT
TCACCCCAATCATCTGTCCCACCTTCGGCGGCTGGCTCCATAAAGGTTAC
CTCACCGACTTCGGGTGTTACAAACTCTCGTGGTGTGACGGGCGGTGTGT
ACAAGGCCCGGGAACGTATTCACCGCGGCATGCTGATCCGCGATTACTAG
CGATTCCAGCTTCACGCAGTCGAGTTGCAGACTGCGATCCGAACTGAGAA
CAGATTTGTGRGATTGGCTTAACCTCGCGGTTTCGCTGCCCTTTGTTCTG
TCCATTGTAGCACGTGTGTAGCCCAGGTCATAAGGGGCATGATGATTTGA
CGTCATCCCCACCTTCCTCCGGTTTGTCACCGGCAGTCACCTTAGAGTGC
CCAACTGAATGCTGGCAACTAAGATCAAGGGTTGCGCTCGTTGCGGGACT
TAACCCAACATCTCACGACACGAGCTGACGACAACCATGCACCACCTGTC
ACTCTGCCCCCGAAGGGGACGTCCTATCTCTAGGATTGTCAGAGGATGTC
AAGACCTGGTAAGGTTCTTCGCGTTGCTTCGAATTAAACCACATGCTCCA
CCGCTTGTGCGGGCCCCCGTCAATTCCTTTGAGTTTCAGTCTTGCGACCG
TACTCCCCAGGCGGAGTGCTTAATGCGTTAGCTGCAGCACTAAAGGGGCG
GAAACCCCCTAACACTTAGCACTCATCGTTTACGGCGTGGACTACCAGGG
TATCTAATCCTGTTCGCTCCCCACGCTTTCGCTCCTCAGCGTCAGTTACA
GACCAGAGAGTCGCCTTCGCCACTGGTGTTCCTCCACATCTCTACGCATT
TCACCGCTACACGTGGAATTCCACTCTCCTCTTCTGCACTCAAGTTCCCC
AGTTTCCAATGACCCTCCCCGGTTGAGCCGGGGGCTTTCACATCAGACTT
AAGAAACCGCCTGCGAGCCCTTTACGCCCAATAAtTCCGGACAACGCTTG
CCACCTACGTATTACCGCGGCTGCTGGCACGTAGTTAGCCGTGGCTTTCT
GGTTAGGTACCGTCAAGGTGCCGCCCTATTTGAACGGCACTTGTTCTTCC
CTAACAACAGAGCTTTACGATCCGAAAACCTTCATCACTCACGCGGCGTT
GCTCCGTCAGACTTTCGTCCATTGCGGAAGATTCCCTACTGCTGCCTCCC
GTAGGAGTCTGGGCCGTGTCTCAGTCCCAGTGTGGCCGATCACCCTCTCA
GGTCGGCTACGCATCGTCGCCTTGGTGAGCCGTTACCTCACCAACTAGCT
AATGCGCCGCGGGTCCATCTGTAAGTGGTAGCCGAAGCCACCTTTTATGT
CTGAACCATGCGGTTCAGACAACCATCCGGTATTAGCCCCGGTTTCCCGG
AGTTATCCCAGTCTTACAGGCAGGTTACCCACGTGTTACTCACCCGTCCG
CCGCTAACATCAGGGAGCAAGCTCCCATCTGTCCGCTCGACTTGCATGTA
TTAGGCACGCCGCCAGCGTTCGTCCTGAGCCATGAACAAACTCTAAGGGC
GAATTCTGCAGATATCCATCACACTGGCGGCCGCTCGAGCATGCATCTAG
AGGGCCCAATCGCCCTAT

microbial compositions contain a mixture of Bacillus and Lactobacillus bacteria, wherein the weight ratio of Bacillus to Lactobacillus ranges from 1:10 to 10:1 (e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1 or 10:1). Preferably, the weight ratio of Bacillus to Lactobacillus is about 1:5.

The Bacillus mixture includes about 10-50% Bacillus subtilis by weight (e.g., about 10%, about 15%, about 20%, about 25%, about 30%, about 35%, about 40%, about 45%, or about 50% by weight). Preferably, the mixture includes about 30% Bacillus subtilis by weight. The Bacillus mixture includes about 10-50% Bacillus amyloliquefaciens by weight (e.g., about 10%, about 15%, about 20%, about 25%, about 30%, about 35%, about 40%, about 45%, or about 50% by weight). Preferably, the mixture includes about 20% Bacillus amyloliquefaciens by weight. The Bacillus mixture includes about 10-50% Bacillus licheniformis by weight (e.g., about 10%, about 15%, about 20%, about 25%, about 30%, about 35%, about 40%, about 45%, or about 50% by weight). Preferably, the mixture includes about 30% Bacillus licheniformis by weight. The Bacillus mixture includes about 10-50% Bacillus pumilus by weight (e.g., about 10%, about 15%, about 20%, about 25%, about 30%, about 35%, about 40%, about 45%, or about 50% by weight). Preferably, the mixture includes about 20% Bacillu pumilus by weight.

The Lactobacillus mixture includes about 10-50% Pediococcus acidilactici by weight (e.g., about 10%, about 15%, about 20%, about 25%, about 30%, about 35%, about 40%, about 45%, or about 50% by weight). Preferably, the mixture includes about 30% to 35% Pediococcus acidilactici by weight. The Lactobacillus mixture includes about 10-50% Pediococcus pentosaceus by weight (e.g., about 10%, about 15%, about 20%, about 25%, about 30%, about 35%, about 40%, about 45%, or about 50% by weight). Preferably, the mixture includes about 30% to 35% Pediococcus pentosaceus by weight. The Lactobacillus mixture includes about 10-50% Lactobacillus plantarum by weight (e.g., about 10%, about 15%, about 20%, about 25%, about 30%, about 35%, about 40%, about 45%, or about 50% by weight). Preferably, the mixture includes about 30% to 35% Lactobacillus plantarum by weight. More preferably the Lactobacillus is present in the mixture in equal amounts by weight. Most preferably the mixtures contains about 33.3% Pediococcus acidilactici by weight, 33.3% Pediococcus pentosaceus by weight and 33.3% Pediococcus acidilactici by weight.

A first preferred Bacillus mixture includes 10% by weight Bacillus licheniformis, 30% by weight Bacillus pumilus, 30% by weight Bacillus amyloliquefaciens and 30% by weight Bacillus subtilis (referred to herein as Bacillus Mix #1). Preferably, the Bacillus subtilis in Bacillus Mix #1 is Bacillus subtilis subsp. Mojavenis.

A second preferred Bacillus mixture includes Bacillus licheniformis, Bacillus pumilus, Bacillus amyloliquefaciens and Bacillus subtilis (referred to herein as Bacillus Mix #2).

A third preferred Bacillus mixture includes Bacillus subtilis 34 KLB (referred to herein as Bacillus Mix #3).

A preferred Lactobacillus mixture includes equal weights of Pediococcus acidilactici, Pediococcus pentosaceus and Lactobacillus plantarum (referred to herein as Lactobacillus Mix #1).

A preferred microbial biocatalyst according to the invention includes at least about 85% by weight of dextrose, about 0.1 to 5% by weight of Bacillus Mix# 1, about 0.1 to 5% by weight of Bacillus Mix# 2, about 0.01 to 2% Bacillus Mix #3 and about 1 to 15% by weight of Lactobacillus Mix #1. Preferably, the microbial biocatalyst according to the invention includes about 0.1 to 4%, 0.1 to 3%, 0.1 to 2% or 0.5 to 1.5% by weight of Bacillus Mix# 1, about 0.1 to 4%, 0.1 to 3%, 0.1 to 2% or 0.5 to 1.5% by weight of Bacillus Mix# 2, about 0.01 to 1%, 0.05 to 1%, 0.05 to 0.5%, 0.05 to 0.4%, 0.05 to 0.3% or 0.05 to 0.2% by weight of Bacillus Mix# 3, and about 1 to 14%, 1 to 13%, 1 to 12%, 5 to 15%, 6 to 15%, 7 to 15% or 8 to 12% by weight of Lactobacillus Mix #1.

Another preferred microbial biocatalyst according to the invention includes about 87.9% by weight of dextrose, about 1% by weight of Bacillus Mix# 1, about 1% by weight of Bacillus Mix# 2, about 0.1% Bacillus Mix #3 and 10% by weight of Lactobacillus Mix #1.

Yet another preferred microbial biocatalyst according to the invention includes about 2.1% a Bacillus mixture by weight, about 10% a Lactobacillus mixture by weight and about 87.9% dextrose by weight. The Bacillus mixture includes about 30% Bacillus subtilis by weight, about 20% Bacillus amyloliquefaciens by weight, about 30% Bacillus licheniformis by weight, and about 20% Bacillus pumilus by weight. The Lactobacillus mixture includes equal amounts of Pediococcus acidilactici, Pediococcus pentosaceus and Lactobacillus plantarum by weight.

The levels of the bacteria to be used according the present invention will depend upon the types thereof. It is preferred that the present product contains bacteria in an amount between about 10⁵ and 10¹¹ colony forming units per gram.

The bacteria according to the invention may be produced using any standard fermentation process known in the art. For example, solid substrate or submerged liquid fermentation. The fermented cultures can be mixed cultures or single isolates.

In some embodiments the bacteria are anaerobically fermented in the presence of carbohydrates. Suitable carbohydrates include inulin, fructo-oligosaccharide, and glucooligosaccharides.

The bacterial compositions are in powdered, dried form. Alternatively, the bacterial compositions are in liquid form.

After fermentation the bacteria are harvested by any known methods in the art. For example the bacteria are harvested by filtration or centrifugation.

The bacteria are dried by any method known in the art. For example the bacteria are air dried, or dried by freezing in liquid nitrogen followed by lyophilization.

The compositions according to the invention have been dried to moisture content less than 20%, 15%, 10%, 9%, 8%, 7%, 6%, 5%, 4%, 3%, 2%, or 1%. Preferably, the composition according to the invention has been dried to moisture content less than 5%.

In some embodiments the dried powder is ground to decrease the particle size. The bacteria are ground by conical grinding at a temperature less than 10 ° C., 9° C., 8° C., 7° C., 6° C., 5° C., 4° C., 3° C., 1° C., 0° C., or less. Preferably the temperature is less than 4° C.

For example the particle size is less than 1500, 1400, 1300, 1200, 1100, 1000, 900, 800, 700, 600, 500, 400, 300, 200, or 100 microns. Preferably, the freeze dried powder is ground to decrease the particle size such that the particle size is less than 800 microns. Most preferred are particle sizes less than about 400 microns. In most preferred embodiments, the dried powderhas a mean particle size of 200 microns, with 60% of the mixture in the size range between 100-800 microns. In various embodiments the freeze dried powder is homogenized.

In various embodiments the bacteria compositions are mixed with an inert carrier such as rice bran, soybean meal, wheat bran, dextrose monohydrate, anhydrous dextrose, maltodextrin, or a mix thereof.

The inert carrier is at a concentration of at least 60%, 70%, 75%, 80%, 85%, 90%, 95% or more. Preferably, the inert carrier is dextrose monohydrate and the inert carrier is at a concentration of about between 75-95% (w/w). More preferably, the dextrose monohydrate is at a concentration of about between 80-95% (w/w), e.g., about between 85-90% (w/w).

In other aspects the bacterial compositions contain an organic emulsifier such as, for example, soy lecithin. The organic emulsifier is at a concentration of about 1%, 2%, 3%, 4%, 5%, 5, 7%, 8%, 9% or 10%. Preferably, the organic emulsifier is at a concentration of between 1% to 5% (w/w).

Further, if desired, the bacterial compositions may be encapsulated to further increase the probability of survival; for example in a sugar matrix, fat matrix or polysaccharide matrix,

Triglyceride containing waste may generally be any stream of waste, bearing at least one triglyceride constituent. The triglyceride containing waste suitable for use with the present invention include, but are not limited to used cooking oil, sludge palm oil, palm, rapeseed, soybean, mustard, flax, sunflower, canola, hemp, jatropha or mixtures thereof.

The triglyceride containing waste is converted into biofuels by combining the waste in a reactor with alcohol and the bacterial compositions of the invention and subjecting the mixture to sonication. Preferably, the alcohol is methanol, ethanol or a combination thereof. Sonication is at 10- 200 kHz (e.g., 10-150 kHz, 10-100 kHz, 20-100 kHz, 20-80 kHz, or 20-50 kHz) for an amount of time sufficient to achieve at least about 80% conversion of triglyceride waste to biodiesel, preferably at least about 85%, 90% or 95% conversion of triglyceride waste to biodiesel. Sonication can be performed for five to thirty minutes, five to twenty minutes, or preferably five to ten minutes. Preferably, the sonication is at 20-100 kHz. The sonication is at room temperature. The volume of triglyceride containing waste material comprises at least 30%, e.g., from 30-95%, 40 to 90% or 50-90% of the useable volume of the reactor. The alcohol concentration is about 5-30% by weight (e.g., about 5-25%, about 5-20%, about 5-15% or about 10-15%) of the triglyceride containing waste. Preferably, the alcohol concentration is about 10-15% by weight of the triglyceride containing waste. The bacterial compositions of the invention are added at about 0.01 to 10%, 0.01 to 5%, 0.01 to 3% or 0.01 to 1.5% by weight of the triglyceride containing waste.

In various embodiments the resulting biodiesel produced by the methods of the invention is washed with water to remove traces of the microbial catalyst and unreacted alcohol.

The compositions or the invention are manufactured by any method suitable or productions of bacterial compositions. Preferably, mixtures of bacteria containing Bacillus and Lactobacillus, are manufactured by individually aerobically fermenting each Bacillus organism; individually anaerobically fermenting each Lactobacillus organism; harvesting each Bacillus and Lactobacillus organism; drying the harvested organisms; grinding the dried organisms to produce a powder combining each of the Bacillus powders to produce a Bacillus mixture; combining each of the Lactobacillus powders in equal amounts to produce a Lactobacillus mixture and combining the Bacillus mixture and the Lactobacillus mixture at a ratio of between 1:10 to 10:1. The Bacillus organisms are Bacillus subtilis, Bacillus amyloliquefaciens, Bacillus licheniformis, Bacillus pumilus, Bacillus meagerium, Bacillus coagulans, and Paenibacillus polymyxa. The Lactobacillus comprises Pediococcus acidilactici, Pediococcus pentosaceus and Lactobacillus plantarum. The mixture has a moisture content of less than about 5%; and a final bacterial concentration of between about 10⁵-10¹¹ colony forming units (CFU) per gram of the composition.

A better understanding of the present invention may be given with the following examples which are set forth to illustrate, but are not to be construed to limit the present invention.

### EXAMPLES

### Example 1

### Preparation of the Microbial Species

The microbes of the present invention are grown using standard deep tank submerged fermentation processes known in the art.

Bacillus Species

Individual starter cultures of Bacillus subtilis, Bacillus amyloliquefaciens, Bacillus licheniformis, and Bacillus pumilus are grown according to the following general protocol: 2 grams Nutrient Broth, 2 grams AmberFerm (yeast extract) and 4 grams Maltodextrin are added to a 250 ml Erlenmeyer flask. 100 mls distilled, deionized water is added and the flask is stirred until all dry ingredients are dissolved. The flask is covered and placed for 30 min in an Autoclave operating at 121° C. and 15 psi. After cooling, the flask is inoculated with 1 ml of one of the pure microbial strains. The flask is sealed and placed on an orbital shaker at 30° C. Cultures are allowed to grow for 3-5 days. This process is repeated for each of the microbes in the mixture. In this way starter cultures of Bacillus subtilis, Bacillus amyloliquefaciens, Bacillus licheniformis, and Bacillus pumilus are prepared.

Larger cultures are prepared by adding 18 grams Nutrient Broth, 18 grams AmberFerm, and 36 grams Maltodextrin to 1 liter flasks with 900 mls distilled, deionized water. The flasks are sealed and sterilized as above. After cooling, 100 mls of the microbial media from the 250 ml Erlenmeyer flasks are added. The 1 liter flasks are sealed, placed on and orbital shaker, and allowed to grow out for another 3-5 days at 30° C.

In the final grow-out phase before introduction to the fermenter, the cultures from thel liter flasks are transferred under sterile conditions to sterilized 6 liter vessels and fermentation continued at 30° C. with aeration until stationary phase is achieved. The contents of each 6 liter culture flask are transferred to individual fermenters which are also charged with a sterilized growth media made from 1 part yeast extract and 2 parts dextrose. The individual fermenters are run under aerobic conditions at pH 7.0 and the temperature optimum for each species:

| Microbe | Temperature Optimum |
|---|---|
| Bacillus subtilis | 35° C. |
| Bacillus amyloliquefaciens | 30° C. |
| Bacillus licheniformis | 37° C. |
| Bacillus pumilus | 32° C. |

Each fermenter is run until cell density reaches 10¹¹ CFU/ml, on average. The individual fermenters are then emptied, filtered, and centrifuged to obtain the bacterial cell mass which is subsequently dried under vacuum until moisture levels drop below 5%. The final microbial count of the dried samples is 10⁹-10¹¹ CFU/g.

Lactobacillus Species

Individual, purified isolates of Pediococcus acidilactici, Pediococcus pentosaceus, and Lactobacillus plantarum are grown-up in separate fermenters using standard anaerobic submerged liquid fermentation protocols at the pH and temperature optimum for each species:

| Microbe | pH Optimum | Temperature Optimum |
|---|---|---|
| Pediococcus acidilactici | 5.5 | 37° C. |
| Pediococcus pentosaceus | 5.5 | 37° C. |
| Lactobacillus plantarum | 5.0 | 35° C. |

After fermentation the individual cultures are filtered, centrifuged, freeze dried to a moisture level less than about 5%, then ground to a particle size of about 100 microns.

The dried bacillus and lactobacillus microbes are combined in equal proportion to give a final dried microbial composition comprising Bacillus subtilis, Bacillus amyloliquefaciens, Bacillus licheniformis, Pediococcus acidilactici, Pediococcus pentosaceus, and Lactobacillus plantarum with a microbial activity between 10⁸ and 10¹⁰ CFU/g.

### Example 2

### Preparation of the Microbial Species Via Solid Substrate Fermentation

The microbial mix of the present invention can also be prepared via solid substrate fermentation according to the following process:

Bacillus Species

Four pounds of Dairy 12% Mineral Mix, 60 lbs. Rice bran, and 30 lbs Soybean meal were added to a jacketed, horizontal mixer with screw auger. Water and steam were added with mixing to obtain a slurry. After mixing for 2 minutes, 300 lbs wheat bran were added to the mixer followed by more water and steam to re-make the slurry. With the mixer temperature controlled to 35-36° C., 4 lbs of a dry microbial mixture comprising Bacillus subtilis, Bacillus amyloliquefaciens, Bacillus licheniformis, and Bacillus pumilus with an initial microbial activity of about 1×10¹⁰ CFU/g, were added. The mixer was closed; temperature adjusted to 34° C., and the contents allowed to mix for up to 4 days. After fermentation, the contents of the mixer were emptied onto metal trays and allowed to air dry. After drying, a product was obtained with microbial count on the order of 10¹¹ CFU/g and less than about 5% moisture.

Lactobacillus Species

A mixed culture of Pediococcus acidilacctici, Pediococcus pentosaceus and Lactobacillus plantarum was fermented under GMP conditions for up to 5 days on a mixture comprised of: 1 part inulin, 2.2 parts isolated soy protein, 8 parts rice flour with 0.25% w/w sodium chloride, 0.045% w/w Calcium carbonate, 0.025% w/w Magnesium sulphate, 0.025% w/w Sodium phosphate, 0.012% w/w Ferrous sulphate, and 29.6% water. Upon completion of fermentation the mixture was freeze dried to a moisture content less than 5%, ground to a particle size below 800 microns and homogenized. The final microbial concentration of the powdered product is between 10⁹ and 10¹¹ CFU/g.

Final Microbial Mix

The dried bacillus and lactobacillus microbes were combined in equal proportion and ground to an average particle size of 200 microns, to give a final dried microbial composition comprising Bacillus subtilis, Bacillus amyloliquefaciens, Bacillus licheniformis, Bacillus pumilus, Pediococcus acidilactici, Pediococcus pentosaceus, and Lactobacillus plantarum with a microbial activity between 10⁸ and 10¹⁰ CFU/g.

### Example 3

### Formulation of the Microbial Catalyst for Biodiesel Production

The following microbial-based Biodiesel production compositions were prepared:

| | Composition 1 | Composition 2 | Composition 3 |
|---|---|---|---|
| Dried Microbial | 13% | | 13% |
| Composition | | | |
| of Example 1 | | | |
| Dextrose monohydrate | 84% | | 10% |
| Maltodextrin | | | 74% |
| Soy Lecithin | 3% | 3% | 3% |
| Dried microbial | | 97% | |
| composition | | | |
| of Example 2 | | | |
| Bacterial Activity | ≧10⁸ cuf/g | ≧10⁸ cuf/g | ≧10⁸ cuf/g |

### Example 4

### Biodiesel Production Process

An ultrasonic continuous Biodiesel production process was designed according to the following general schematic:

The reactor comprises 304 stainless steel with dimensions of 1.2×2.3 m² and is fitted with inlet ports for methanol, waste oil, and catalyst addition and outlet ports for the Biodiesel/Glycerol mix. An ultrasonic generator operating between 20-100 kHz is used to generate cavitation in the reactor.

The reactor is charged with sludge palm oil at a level between 80-90% of the total useable reactor volume. Methanol is added at between 10-15 wt % of the waste oil. Composition 1 of Example 3 is added at 0.5-1.5 wt % of the total mix. This mixture is then subjected to sonication for 5-10 minutes at room temperature. After sonication the reactor is emptied and the resulting glycerol/biodiesel mix allowed to separate. Significant (+95% yield) Biodiesel production is achieved within 5-10 minutes only when sonication and microbial catalyst are combined:

| | Microbial Catalyst | Sonication | Biodiesel Yield at 10 mins. |
|---|---|---|---|
| Run 1 | No | Yes | 0 |
| Run 2 | Yes | No | 0 |
| Run 3 | Yes | Yes | +95% |

### Example 5

### Expanded Microbial Catalyst Composition

A composition comprising the bacterial strains from Example 1 and additional microbes selected for their ability to provide additional catalytic conversion of waste oil to biodiesel was designed using a fermentation system similar to that developed in Example 1:

Bacillus and Paenibacillus species

Individual starter cultures of Bacillus subtilis, Bacillus amyloliquefaciens, Bacillus licheniformis, Bacillus pumilus, Bacillus coagulans, Bacillus megaterium, and Paenibacillus polymyxa are grown according to the following general protocol: 2 grams Nutrient Broth, 2 grams AmberFerm (yeast extract) and 4 grams Maltodextrin are added to a 250 ml Erlenmeyer flask. 100 mls distilled, deionized water is added and the flask is stirred until all dry ingredients are dissolved. The flask is covered and placed for 30 min in an Autoclave operating at 121° C. and 15 psi. After cooling, the flask is inoculated with 1 ml of one of the pure microbial strains. The flask is sealed and placed on an orbital shaker at 30° C. Cultures are allowed to grow for 3-5 days. This process is repeated for each of the microbes in the mixture.

Larger cultures are prepared by adding 18 grams Nutrient Broth, 18 grams AmberFerm, and 36 grams Maltodextrin to 1 liter flasks with 900 mls distilled, deionized water. The flasks are sealed and sterilized as above. After cooling, 100 mls of the microbial media from the 250 ml Erlenmeyer flasks are added. The 1 liter flasks are sealed, placed on and orbital shaker, and allowed to grow out for another 3-5 days at 30° C.

In the final grow-out phase before introduction to the fermenter, the cultures from thel liter flasks are transferred under sterile conditions to sterilized 6 liter vessels and fermentation continued at 30° C. with aeration until stationary phase is achieved. The contents of each 6 liter culture flask are transferred to individual fermenters which are also charged with a sterilized growth media made from 1 part yeast extract and 2 parts dextrose. The individual fermenters are run under aerobic conditions at pH 7.0 and the temperature optimum for each species:

| Microbe | Temperature Optimum |
|---|---|
| Bacillus subtilis | 35° C. |
| Bacillus amyloliquefaciens | 30° C. |
| Bacillus licheniformis | 37° C. |
| Bacillus coagulans | 37° C. |
| Bacillus megaterium | 30° C. |
| Bacillus pumilus | 32° C. |
| Paenibacillus polymyxa | 30° C. |

Each fermenter was run until cell density reached 10¹¹ CFU/ml, on average. The individual fermenters were then emptied, filtered, and centrifuged to obtain the bacterial cell mass which was subsequently dried under vacuum until moisture levels drop below 5%. The final microbial count of the dried sample was 10¹⁰-10¹¹ CFU/g.

Lactobacillus Species

The lactobacilli; Pediococcus acidilactici, Pediococcus pentosaceus, and Lactobacillus plantarum, were grown according to the protocol outlined in Example 1.

The dried bacillus and lactobacillus microbes are combined in equal proportion, ground and homogenized so that average particle size is 200 microns, to give a final dried microbial composition comprising Bacillus subtilis, Bacillus amyloliquefaciens, Bacillus licheniformis, Pediococcus acidilactici, Pediococcus pentosaceus, and Lactobacillus plantarum with a microbial activity between 10⁸ and 10¹⁰ CFU/g.

We claim:

1. A method of converting triglyceride containing waste into biodiesel comprising:
a. combining in a reactor(i) triglyceride containing waste, (ii) methanol and (iii) a microbial biocatalyst comprising a mixture of Bacillus and Lactobacillus organisms and
b. subjecting the resulting mixture to sonication.

2. The method of claim 1 wherein the triglyceride waste is derived from used cooking oil, sludge palm oil, palm, rapeseed, soybean, mustard, flax, sunflower, canola, hemp, jatropha or mixtures thereof.

3. The method of claim 1, wherein the Bacillus organisms are a mixture of Bacillus subtilis, Bacillus amyloliquefaciens, Bacillus licheniforms, and Bacillus pumilus,

4. The method of claim 3, wherein each of the Bacillus in the mixture is individually aerobically fermented, harvested, dried, and ground to produce a powder having a mean particle size of about 200 microns, with greater than about 60% of the mixture in the size range between 100-800 microns.

5. The method of claim 1, wherein the Lactobacillus organisms are a mixture of Pediococcus acidilactici, Pediococcus pentosaceus, and Lactobacillus plantarum.

6. The method of claim 5, wherein each of the Lactobacillus in the mixture is individually anaerobically fermented, harvested, dried, and ground to produce a powder having a mean particle size of about 200 microns, with greater than about 60% of the mixture in the size range between 100-800 microns.

7. The method of claim 3, wherein the Bacillus organisms further comprise a mixture of Bacillus megaterium, Bacillus coagulans, and Paenibacillus polymyxa.

8. The method of claim 7, wherein each of the Bacillus in the mixture is individually aerobically fermented, harvested, dried, and ground to produce a powder having a mean particle size of about 200 microns, with greater than about 60% of the mixture in the size range between 100-800 microns.

9. The method of claim 1, wherein the ratio of the Bacillus to Lactobacillus is between 1:10 to 10:1.

10. The method of claim 1, wherein the microbial biocatalyst comprises about 87.9% by weight of dextrose, about 1% by weight of Bacillus Mix# 1, about 1% by weight of Bacillus Mix# 2, about 0.1% Bacillus Mix #3 and about 10% by weight of Lactobacillus Mix #1.

11. The method of claim 1, wherein the microbial catalyst comprises about 2.1% a Bacillus mixture by weight, about 10% a Lactobacillus mixture by weight and about 87.9% dextrose by weight.

12. The method of claim 11, wherein the Bacillus mixture comprises 30% Bacillus subtilis by weight, about 20% Bacillus amyloliquefaciens by weight, about 30% Bacillus licheniformis by weight, and about 20% Bacillus pumilus by weight and the Lactobacillus mixture includes equal amounts of Pediococcus acidilactici, Pediococcus pentosaceus and Lactobacillus plantarum by weight.

13. The method of claim 1, wherein the microbial biocatalyst has a moisture content of less than about 5%; and a final bacterial concentration of about between 10⁵-10¹¹ colony forming units (CFU) per gram.

14. The method of claim 1, wherein the microbial biocatalyst further comprises an inert carrier.

15. The method of claim 14, wherein the inert carrier is rice bran, soybean meal, wheat bran, dextrose monohydrate, maltodextrin, or a mix thereof.

16. The method of claim 14, wherein the inert carrier is at a concentration of about 75-95% (w/w).

17. The method of claim 1, wherein the microbial biocatalyst further comprises an organic emulsifier.

18. The method of claim 17, wherein the organic emulsifier is at a concentration of about between 1 to 5% (w/w).

19. The method of claim 17, wherein the organic emulsifier is soy lecithin.

20. The method of claim 1, wherein the volume of triglyceride containing waste material comprises from 50-90% of the useable volume of the reactor.

21. The method of claim 1, wherein the methanol concentration ranges from 10-15% by weight of the triglyceride containing waste material.

22. The method of claim 1, wherein the microbial catalyst is added at 0.01 to 1.5% by weight of the triglyceride containing waste material triglyceride containing waste material.

23. The method of claim 1, wherein sonication is conducted for 5-20 minutes.

24. The method of claim 1, wherein the resulting biodiesel is washed with water to remove traces of the microbial catalyst and any unreacted methanol.

25. A composition comprising about 2.1% a Bacillus mixture by weight, about 10% a Lactobacillus mixture by weight and about 87.9% dextrose by weight, wherein the Bacillus mixture comprises about 30% Bacillus subtilis by weight, about 20% Bacillus amyloliquefaciens by weight, about 30% Bacillus licheniformis by weight, and about 20% Bacillus pumilus by weight, and wherein the Lactobacillus mixture comprises equal amounts of Pediococcus acidilactici, Pediococcus pentosaceus and Lactobacillus plantarum by weight.
